(19) United States
(12) Patent Application Publication
(10) Pub. No.: US 2012/0179003 A1
(43) Pub. Date: Jul. 12, 2012

(54) Therapeutic Treatment Systems and Processes for Stress-Related Disorders

(75) Inventor: D. C. Faber, Laramie, WY (US)

(73) Assignee: FABER GROUP SYNERGY, LLC, Laramie, WY (US)

(21) Appl. No.: 13/348,518

(22) Filed: Jan. 11, 2012

Related U.S. Application Data
(60) Provisional application No. 61/432,073, filed on Jan. 12, 2011.

(51) Int. Cl. A61B 5/00

(52) U.S. Cl. 600/300

(57) ABSTRACT

A process and automated computer process implemented system are provided to transform therapy and intercranial processes as an aid in therapeutically treating incidents of stress-related disorders such as PTSD or other aspects. Effects may be indicated and assessed by determining a level of concentrated emotional presentness and/or a level of emotional intensity or the like. Various embodiments can provide guidance to both subjects and counselors and can automatically indicate progress or optimal therapies to be used so that a subject, perhaps including but not limited to combat veterans and others who have experienced or been subjected to a stressful situation perhaps characterized by a fight or flight basal reaction. Through the system, subjects can be encouraged to obtain and continue treatment and may more rapidly progress with treatment by being aware of a level of emotional presentness and/or emotional intensity that are incidents of intense stress-related experiences.

### CROSS-REFERENCES TO RELATED APPLICATIONS

This is a United States Nonprovisional application claiming the benefit of and priority to U.S. Provisional Application No. 61/432,073, filed Jan. 12, 2011, hereby incorporated by reference.

### TECHNICAL FIELD

This invention relates initially to system beneficial to those suffering from stress-related disorders such as post-traumatic stress disorder, disorders related to the threat of trauma, and the like. It has ramifications and can be expanded to other systems related to treatment as well as present an overall system to enhance or encourage participation in treatments for those suffering from such disorders such as veterans and others similarly afflicted. More specifically, it involves systems which may be configured as pre- and post-stress events. For post-stress events, it may be configured in a therapeutic and treatment enhancement modality. For pre-stress events, it may be configured in a preparatory and education modality to reduce the effects during and after such events. It is particularly applicable to service men and women involved in armed service and/or in actual harm's way. The invention is particularly helpful to returning veterans who may be experiencing post-traumatic stress disorder (PTSD) as well as others who are experiencing symptoms as a result of stress.

### BACKGROUND

PTSD is a disorder that has been more recent a matter of focus and understanding. Approximately 10-20% of the two million U.S. troops who have served in the wars in Afghanistan and Iraq experience significant mental health difficulties including PTSD, depression, and anxiety. There are various treatment methods currently in use. However none addresses the symptoms and experiences occurring of many veterans. Further, significant numbers of Veterans do not experience formal psychopathology but do experience difficulties in normative reintegration into society. The present method expands upon existing treatments and presents new treatments and processes that were developed by a serviceman having actually served in combat and in the field in Operation Enduring Freedom, and who has actually experienced first-hand the effects war can inflict upon those serving in and returning from stressful environments.

### SUMMARY DISCLOSURE OF INVENTION

The present invention provides technology and methods and apparatus that can enhance existing treatments and can present additional treatments and processes for those having been subjected to stress or a stress-like situation. It presents a process that can use new modeling structures and understandings to treat such disorders. From some perspectives, it is even surprising that a system could be developed and presented by practical and personal experience with the symptoms and effects of such a disorder. Through various embodiments, the present invention shows that various processes and devices that can be provided to enhance treatments, to present new treatments, to present a framework from which new treatments can be developed, to identify ways to intervene as soon as feasible to prevent spiraling dysfunction, suffering, premature discharge, and chronic problems for those afflicted, and even to aid in normative reintegration difficulties for a broader spectrum of military personnel. Naturally, further objects of the inventive technology are apparent from the description and drawings below.

### BRIEF DESCRIPTION OF THE DRAWINGS

FIG. 1 is a schematic diagram of a process sequence according to one embodiment.

FIG. 2 is a graphic diagram of some initial parameters that may be used in one embodiment of the invention.

FIG. 3 is conceptual diagram of one model of a communication pathway such as may be considered in understanding embodiments of the present invention.

FIG. 4 is a schematic diagram of a device utilizing a therapy aid according to one embodiment.

FIG. 5 is conceptual diagram of a visual image that may be used in communicating understandings as may be used throughout embodiments of the present invention.

FIG. 6 is a sample self-evaluation form indicative of temporal presentness.

FIG. 7 is a sample self-evaluation form indicative of emotional intensity

FIG. 8 is a sample presentation form showing changes over time.

### MODES FOR CARRYING OUT THE INVENTION

As mentioned earlier, the present invention includes a variety of aspects, which may be combined in different ways. The following descriptions are provided to list process steps and to describe some of the embodiments of the present invention. These elements are listed with initial embodiments, however it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed processes or elements, with each process or element alone, and also with any and all various permutations and combinations of all processes or elements as may be presented in this or any subsequent application.

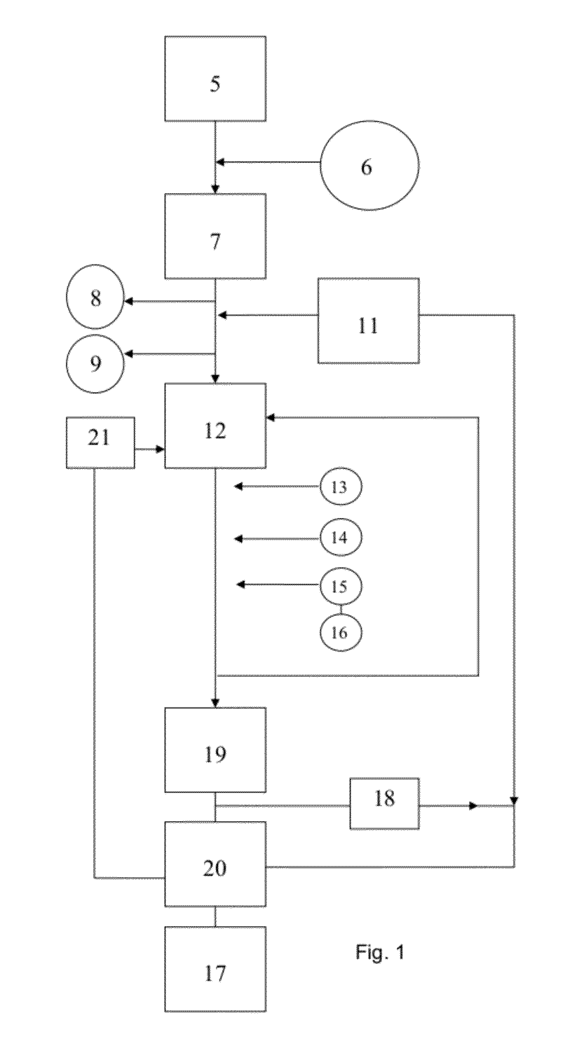

FIG. 1 presents a schematic diagram of an embodiment of the invention. It provides a context to step through various therapy steps involved, and to describe aspects of the present invention. To understand a context for this treatment embodiment, it is first helpful to understand that there is sometimes a core assumption for some in the physiological community that veterans and severe trauma patients may be primarily emotionally focused in the past rather than the present. The present FPTM model considers and at times illustrates the opposite, namely, that some can be almost exclusively focused in the present. This can present a significant difference in emotional orientation that may be critical for understanding, treatment, and/or growth from the war/injury experience.

One embodiment may be considered as developed from a model termed the Faber Post-Trauma Model (FPTM) that describes what can sometimes happen to human beings who experience trauma or even the threat of trauma. Each of these can be more generally considered as a stress event from which there may be long term impacts. In one model there may be separate phases or events that may be considered as occurring:
First, a subject may experience a traumatic event or even the threat of trauma or other stress.
Second, this event can be considered as potentially causing or may unquestionably cause the brain to shift from a normal function mode to start operating in a more protective mode, perhaps the survival mode, such as the primitive/survival/fight-or-flight function mode (survival oriented brain functioning, e.g., more amygdalar activation) or to operate from that sector of the brain. It may be considered that the reasoning, perhaps frontal lobe, functionalities of the brain may generally not be the part of the necessary brain function during flight/fight/freeze response. It is noteworthy that in some models, the frontal lobe is generally identified as the part of the brain that also registers hope and emotional connection to the future. From some understandings, when the survival mode of the brain is primarily operative, the individual may no longer be fully able to experience a sense of hope or happiness unless in the immediate time frame.
Third, since the brain is operating in what may be considered its survival mode, the individual may be more aware of present stimuli; the individual's own emotional/mental focus may be directed toward the present, such as what may be life threatening or dangerous.
Fourth, this emotional presentness can cause a concentration of a variety of emotions condensed solely into the present. There may be no emotional need for events past or future, so the individual may experience an intensity of emotion, and may feel the extreme range of a given emotion. Contrary to any assumptions of emotional numbness, the present emotion may actually be intensified, as if not spread out over time, to a level not typically common in those not so affected.
Finally, if the situation is a matter of life or death, an individual may have to or may actually accept that they will die, someone else will die, or someone else did actually die. This can be attended by experiencing grief perhaps such as Kübler Ross's Grief Cycle. If the individual lives through the trauma, they may surprisingly have to experience Kübler-Ross's Grief Cycle yet again to accept that they, indeed lived. They may have to accept living after the death of others as well. Emotions and perhaps even brain function after having accepted one's own likely death can be an entirely new concept, or can at least take on a different character, which may separate one embodiment of the present invention and even one post-trauma model from others. The FPTM can be used in the invention to make a distinction between psychological trauma and stress such that individuals do not catastrophize or accentuate emotional responses to a mere stress by virtue of having a trauma history. Concrete suggestions for responding more effectively to mundane stressors (e.g. leaving the situation, taking a walk, etc.) may be encouraged in order to illuminate behavioral choices that can de-escalate a stressful situation. The invention can also serve as an educational tool designed to educate Veterans, supportive others and community members about common difficulties encountered by Veterans when reintegrating after deployment.
An initial aspect for consideration may be that of the stress itself. This can be present in either trauma or even the threat of trauma. Post Traumatic Stress Disorder (PTSD) is widely studied and the present invention can utilize a model that need not challenge any of those existing ideas or suggestions. In some embodiments, the trauma modeling can be used to address and even illustrate the differences between trauma, and the threat of trauma. Further, the model may be used in ways which may be similar to some PTSD studies or may be unique in the perspective of embodiments of the present invention.
Trauma may cause some individuals to later experience similar reactions to similar trigger stimuli even after the trauma has taken place. Some individuals can notice a reaction to trauma or even the threat of trauma or stress long after the initial event (susceptibilities can vary). For example, items such as loud noises, gun shots, traffic, crowds, familiar smells, or even someone who looks like the one who caused the trauma, can result in a reaction. Emergency Medical Technicians (EMTs) are taught the acronym SAMPLE when arriving on-scene and needing to make a patient assessment. The “S” stands for signs/symptoms of what an EMT may see of the patient even before they touch or make contact with the patient. There may be a visual assessment and an EMT may need to look for obvious injuries, bleeding, confusion, lack of breathing, etc. The remainder of the acronym is not necessary to discussion of this initial “signs/symptoms” aspect and will not be outlined. However, the signs/symptoms aspect is one way some embodiments can be used to distinguish a noticeable difference between trauma and the threat of trauma or other stress when it happens. It can also aid in determining how the event(s) is/are experienced and perceived during and after the event(s).

Whatever trauma an individual underwent can also be experienced again in response to certain, perhaps similar, triggers even after the event. For instance, a soldier may jump when rocketed/mortared, and loud noises can cause that same response after the event. There are many physical and emotional reactions that occur externally that happen internally which may go unnoticed unless the patient is assessed more thoroughly. There are too many examples to list, but many follow the same formula. An event occurred in the past, and a current event or stimuli triggers a current reaction. Importantly, this can be noticed and used in embodiments of the present invention whether or not associated with a formal psychological disorder or formal psychopathology (e.g., PTSD, depression, substance abuse, etc.)

The scale of reactions can be caused by, but is not limited to, the degree of the past or present event(s), the number of instances, the degree of violence or intensity, the length of time over which the trauma may occur, and/or how often or if the trauma was experienced repeatedly. Again, embodiments need not presume to limit the amount of utilization of such factors or even state exact degrees of experience as such inputs or considerations. Much study has already been undertaken on these subjects. Some embodiments may use a model that simply states that many people display future signs and symptoms of trauma, long after the event.

The threat of trauma or stress event in a specified environment can be similar to trauma with regard to degree: the number of instances, the degree of violence, the length of time over which the moment of the trauma may occur, or how often they are repeated. All of these can be factors in models used in some embodiments. Again, embodiments need not intend to limit the amount or state exactly what those degrees may be. It may be noted that the threat of trauma or stress can sometimes be more difficult to identify or observe both for treatment participants as well as the subject themselves. This can be compounded by a common mis- or other perception that without first hand experiencing a traumatic event reactions or affectations are less likely or nonexistent. The present invention shows that this is not always accurate. An individual in combat may seem to be fine, they are surviving, eating, engaging others, but underneath—whether consciously or even subconsciously—they may be experiencing constant life or death feelings or realizations. For example, consider events or activities such as: making decisions about missions to be conducted, endangering themselves and their men/women, standing guard in towers/at gates, serving as a gunner in a vehicle. This list is not exhaustive by any means, but importantly, each can trigger effects even though not trauma or traumatic event is experienced or actually occurs. Another example in other than a war environment concerns individuals such as those who were raised in violent homes. It is the stress surrounding the anticipation of the perhaps inevitable violence, which can be as destructive to the psyche as the violence itself and the present invention utilizes and applies this understanding in manners that are unique.

An embodiment or model applicable to the invention can utilize the fact, and show or suggest, that there is a difference between trauma and the threat of trauma or stress that may be significant. Trauma can be considered an event with a beginning and an end. The threat of trauma or such stress can last long. It can exist independently of a trauma or it can be considered as encompassing the remainder of the time between or surrounding the traumatic event(s). It can even at times, seem endless. Importantly, it should be understood that the threat of trauma can be experienced in both safe and unsafe environments. The perception of safety, or lack thereof, may even be directed or created by the individual. It may result in the individual operating on a hyper-vigilant level to always determine the level of safety. The degrees of a threat of trauma may also compound how and when an individual perceives they are safe in the future. If the individual perceives the environment is not safe, the brain can react to that stimuli or perception and can revert to functioning largely in survival mode even without an actual present threat.

A second aspect that may be considered is that of brain function, such as a perspective that when an individual experiences trauma or a threat of trauma, normal function, perhaps frontal lobe function, may be diminished to aid in basic survival. The brain may be considered as operating primarily from perhaps a primitive, or even “reptile” sector. The brain may resort to survivalist functioning such as the fight/flight/freeze reaction. As a result, an individual who is experiencing trauma, a threat of trauma, or even being similarly triggered to such an event, may not need a fully functional reasoning capability (perhaps such as found in the frontal lobe) in order to survive. As a result, the individual may experience a change in how they engage their environment and h they operate at that moment.

Dr. Daniel Gilbert, the Harvard psychologist and the author of “Stumbling on Happiness” has a hypothesis which states: “The human being is the only animal that thinks about the future.” He goes on to explain that the part of the brain that gives us this ability is the frontal lobe. From this perspective, the frontal lobe can be considered the part of the brain that allows us to perceive the future and to connect emotionally to future events.

One embodiment may involve the perspective that individuals like veterans and trauma victims may be considered metaphorically or otherwise as having a frontal lobe injury, impairment, or need. As a result these individuals may not have the capacity to emotionally connect to the future. These individuals may only be minutes to hours out in their ability to experience emotional connection to anything beyond that time boundary. The amount of time forward someone can think and also experience emotion presently can vary depending upon the degrees of trauma or the threat of trauma or other stress experienced or triggered as previously explained. Conversely, subjects may feel more of an emotional connection to their planning efforts, if those efforts are closer to immediate needs, wants, or desires.

A model used in some embodiments uses a combat veteran as an example. Not all combat veterans may have a frontal lobe injury or impairment, but it seems many do, as has been the present inventor's experience personally and in speaking with countless vets. For example, during a traumatic event, such as being shot at or bombed, our brain orientation can shift to the survival/primitive part of the brain so the body can survive. The brain may stay engaged during trauma and can be almost permanently locked into this survival orientation if the trauma or threat of trauma or other stress is long enough or intense enough. The frontal lobe or reasoning capability can even be considered as of little use in a hostile/traumatic environment and its capacity to fully function can be altered at that time. The frontal lobe may reduce its functioning to an extremely low level of operation, even after an individual is removed from the trauma/stress of trauma space.

It can be considered that time can heal such disorders as well. The longer an individual is removed from a traumatic environment there seems to be a correlation to how far out one can think and feel emotionally connected to that thought about the future. Embodiments of the invention can accelerate this healing as well.

Future happiness and hope can be very difficult for many traumatized individuals or threat-of-trauma impacted individuals, perhaps because the frontal lobe can be considered injured from either the actual trauma or the threat of trauma. Therefore, hope may only register as far out as the survival part of the brain can register safety. Additionally, since there can be considered an injury or impairment to the frontal lobe or some portion of the brain, there can be little or no fear of, or worry about, the future. Needs can be mostly in the immediate and that can be considered as explaining why so many veterans or trauma victims often make compulsive decisions to meet an immediate need without fear of consequence. Tomorrow, or even later today, may not be considered important or relevant. There can be very little, if any, connection emotionally and therefore mentally. Many people with this injury, who are otherwise mature and capable, live in the “now,” and can tend to make decisions based on instant gratification, rather than rational thought perhaps as if their frontal lobe or other functioning is damaged.

Another attribute that may be utilized in embodiments of the present invention may be the aspect termed Concentrated Emotional Presentness. Concentrated Emotional Presentness can be the connection to this moment; this now moment. Subjects with an impaired level of emotional presentness, or a Concentrated Emotional Presentness, may commit no mental or emotional energy to the past, nor may any be committed, with any significant duration, to the future. The degree of trauma can be considered as proportionate to the degree of presentness (e.g., a one-time event like car crash where one observes death, vs. a firefighter responding to traumatic calls over a 20-year career, vs. a Veteran (noncombat/combat/multiple tours) vs. a child brought up in a violent and volatile household). Any of these degrees of trauma can transform the person so they only put energy into the now; the longer or intense the trauma, as actual or perceived, the more pervasive the now tendency or moments. Any stimulation that reminds the person of the trauma or the threat of trauma (stress), can cause many people to immediately become emotionally and/or mentally present, similar to when the trauma first took place. This can delay the healing effects of time as well.

Another concept that can be used in embodiments of the invention is that of Emotional Intensity. As used in embodiments, Emotional Intensity can be considered as the strength of feeling an emotion in this now moment. This can be coupled with the concept of emotional energy, such as the momentary intensity integrated over the duration of that intensity. For many non-traumatized individuals, their emotional energy can be considered as stretched and lessened in intensity or even softened because the emotional energy is mentally connected to and spread over the past, the present, and projected into the future. In normal individuals, it can be considered that one's whole life is constantly utilized as the emotional scale the person registers, and throughout which the emotional energy can at times be spread. A traumatized individual, who has Concentrated Emotional Presentness, and who may not necessarily be capable of stretching out their emotional energy to the past or projecting it into the future, may experience and be subjected to all the emotional energy and experiences in the present moment. Duration can shorten and intensity can correspondingly rise.

Differing embodiments of the invention can utilize an assumption that human capacity for emotion is equal for everyone or can vary by individual, but either way they can consider that energy as compressed to the present. They can utilize the perspective that the volume of emotion is the same, however because it is temporally compressed, its intensity can be heightened in the moment. Whether the person who is emotionally intense or not, a person with the disorder may notice their level of emotion in the moment (anger, happiness, and depression) to be heightened.

Yet another aspect that can be utilized in some embodiments of the invention can be that mentioned earlier relative to situations of life or death where an individual may consciously or subconsciously accept that they will die. Some traumatized individuals who may have had to accept their own death, death of others, or that death was imminent for themselves or others, may be considered as encountering a functional shift in their brain. This shift can be considered as potentially transforming intercranial functionality, or effecting (actually or metaphorically) a frontal lobe transformation, from a “normal” function to one that operates more significantly in a survival mode. The individual may be considered as operating out of the perhaps primitive/reptile/survival portion of their brain. Importantly, this shift can be considered as a possibly permanent or semi-permanent change, or even as an injury. Knowing one is going to die, or accepting one's own or another's death can be considered as if causing a switch in the manner in which the brain functions. Embodiments of the invention can utilize a perspective such as a switch that occurs intercranially. For the brain and perhaps even the rest of the body, crossing a death realization threshold can be considered as causing a change in function and even an injury. This intercranial switching event can also be triggered if one witnesses others die, or is the cause of another's death. Brain functioning—and the person—may never be the same.

Embodiments of the invention can present processes that are different from any other system, or even model, by utilizing this important insight in yet another fashion. For example, embodiments may present processes that are founded upon a consideration that one way in which to heal and start fully living again is to present processes that can also acknowledge and go through a grief event or grief cycles such as Kübler-Ross's grief cycle, twice. A first grief cycle can be associated with accepting death, then a second grief cycle can be associated with accepting that one did not die, but rather survived. This second cycle can involve processes to re-connect with life and all that that entails. Individuals may even go through the grief cycle many times; grieving many losses or many stress events, such as experiencing the stress event of accepting the likelihood of one's own death, experiencing the stress event of accepting that one did not die, experiencing the stress event of having another die, experiencing the stress event of having involvement in such moments, experiencing the stress event of a threat of trauma, and the like. A challenge that pervades the counseling community is that few recognize the toll exacted on the brain and body in accepting one's own or another's death and the like. Embodiments of the invention can utilize processes that explain why the subject must accept they did, indeed, live, may present processes that address that without addressing such intercranial injury, the experience of living may seem one of confusion, or perhaps still in trauma, or worse. The experience of not accepting that one lived can be evidenced in phrases like, “I was supposed to die over there”, “I should have died over there” or “why did I live and my friends didn't?” Embodiments present processes that address these as a fundamental aspect to achieving the required transformation of the injured intercranial functionality.

Currently, there appear to be no scientific models which so expand Kübler Ross's Grief Cycle model or other grieving therapies in the way of embodiments of the present invention or that advocate and address the necessity of accepting life after having accepted death as does the present invention. Additionally, there appears to be little or perhaps no processes that address or seek to provide treatment from the perspective of reversing or accelerating healing of the disorder's intercranial functionality transformation or switch when one returns to environments that are safe from abuse or trauma. The functional switching can happen in the brain in a life or death traumatic moment, and embodiments of the present invention can use and recognize that there may be no event or re-transformation process naturally occurring in safe environments that can be considered as re-engaging the frontal lobe or undoing the harmful transition or switch to cause the brain to function normally again. Embodiments of the invention can present processes and systems through which and which may help individuals understand what happened mentally, emotionally, and physically and may help to re-transform the mental processes or intercranial functionalities back to normal. Embodiments may also be presented that can aid in leading the individual to recognize when they are in safe environments, and so may allow the brain functions to be less likely to retrigger and more likely to slowly return to normal function. Embodiments can cause, aid, speed, or enhance a transformation of the associated intercranial functions (actually or metaphorically) back to normal. Embodiments of the invention can serve to assist in achieving more functional outcomes for Veterans, to enhance support for Veterans by significant others, and to facilitate understanding and awareness of challenges experienced by Veterans returning home after combat and others.

In embodiments so configured, accepting death can be considered as a threshold that, once crossed, may be treated through systems or processes that address this threshold event as one that may result in permanent or semi-permanent injury that may be very difficult to come back from functionally and even emotionally. In embodiments, re-accepting life can also be considered a parallel threshold. This, too, may be treated as an injury causality and as a threshold that one may need to cross over in order to let the brain and body function correctly and even live again.

In embodiments, therapies can involve systems that aid in counseling processes that can be helpful at any point in the methods when the knowledge of the model is properly understood. Another potential outcome or goal for some embodiments can even be simply that more individuals will be receptive to, participate in, or be positively affected by counseling if all parties involved have a common understanding of the individual's emotional and mental orientation, mental functional transformation, intercranial injury, or even sense of presentness. Embodiments can even be such that merely the drop-out treatment rate for individuals who have experienced trauma or a threat of trauma or stress will substantially decrease.

With the above contexts, it can be understood how embodiments can present differing systems, differing therapies, and differing treatment processes. Referring to FIGS. 1 & 2, and to the potential definitions of embodiments of the invention, it can be understood that embodiments may utilize a variety of steps. An initial step in the overall processes of some embodiments can involve establishing an external condition (5) that may be conducive to either transformation of an intercranial communication pathway (1) or perhaps to an enhanced rate of development of an intercranial communication pathway (1). As may be understood the concept of an intercranial communication pathway (1) can be conceptual in nature. It should be understood as encompassing the possibility of an actual communication pathway as well as merely a functionality operability characterized as an intercranial communication pathway (1).

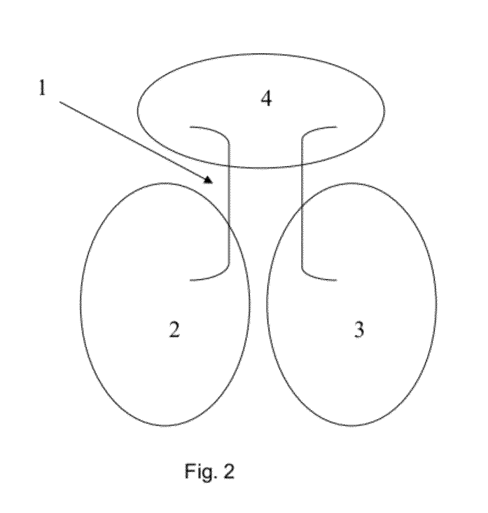

As shown in FIG. 2 at least conceptually, the mental functions may be understood in the context of a communication framework. This communication framework may be considered as involving a variety of mental regions or perhaps functionalities (the two being alternative considerations or merely constructs). These regions or functionalities may include a left hemispheric lobe or construct (2), a right hemispheric lobe or construct (3), and perhaps a frontal lobe or construct (4). The frontal lobe (4) may be a region or functionality that applies reasoning. As may be appreciated, it is a left hemispheric lobe (2) or the right hemispheric lobe (3) that may be considered as the functioning part for survival mode mental functionality or operations. It can be considered that a communication pathway, specifically perhaps the conceptual intercranial communication pathway (1), as shown, can be impaired, diminished, or even injured as a result of the stress or trauma event. Whether a functionality or an actual pathway, it can be considered as this is the feature which needs to be transformed to again present a fully functioning state. This transformation may occur slowly or not at all. It may need the enhancement or encouragement offered by the present invention to transform or to transform more quickly. Thus, aspects of the invention can either cause the transformation or at least accelerate it.

Referring again to FIG. 1, the processes can involve some or all of a variety of steps achieved by or as components of a system of the invention. As shown, embodiments may include processes such as the steps of (or, in apparatus embodiments, as elements for-) establishing an external condition (5), assessing an initial parameter (7), administering a stress related disorder therapy (12), and transforming an intercranial communication pathway or the like (17). As shown in FIG. 1, a variety of other steps may or may not also be included.

Stepping through FIG. 1, it can be appreciated that an initial step can involve establishing an external condition (5) conducive to an enhanced rate of, or to the existence of, an intercranial communication development or functionality modeled as an intercranial communication pathway (1)(shown in FIG. 2). A step can involve developing for the subject a temporal awareness paradigm such as by establishing an awareness in the subject of an emotional presentness (6). This awareness can aid in the base or other therapy. An aspect that can also aid in the therapy can be the step of assessing an initial parameter (7). This assessment can be of a variety of parameters, and included may be a parameter such as the step of determining a temporal presentness level (8) which may be indicative of the intercranial communication pathway (1) or functionality or the like. Similarly, embodiments can involve determining or even developing an emotional intensity modeling level (9). This emotional intensity can even be developed and the level determined from an indication of temporal presentness such as may exist if the step of assessing a concentrated emotional presentness (26) is accomplished. In some embodiments, the two may be considered as interrelated, one can be used to estimate or determine the other and the like.

As shown in FIG. 1, an additional step which may be included in embodiments can be the step of recording subject treatment-precedent information (11). This step can include recording treatment-precedent demonstrative information such as subjective or objective responses that can be used to determine or provide information to determine or estimate a level of emotional presentness for the particular subject. This can include quantitative information. It may also involve subjective information perhaps such as the number of times particular words of the like occur in spontaneous verbalization. As can be appreciated and as discussed later on. This recorded information can be utilized to validate, compare, or even refine and adjust administered therapies.

Naturally an important part of the process can involve administering a stress-related disorder therapy (12). This can occur at any effective level and can involve any type of particular therapy. The therapy can involve counseling (13), pharmaceutically involved therapies (14), and other therapies as may already exist or subsequently be developed in the art and as disclosed in some of the references incorporated by reference. Within the scope of the present invention, either known or otherwise developed therapies can be used and/or the therapy sequences set out in this disclosure can be used. All otherwise known therapies are available and some of the possibilities are set forth in references listed later in this disclosure and hereby incorporated by reference. As can be seen from the therapies in these references, a broad possibility is available from process steps involving counseling alone or with other processes, to pharmaceutically-derived therapies, to even seemingly simplistic self administered therapies. Although it is anticipated that those therapy processes discussed in specific here are or may prove to offer the best advantages, it should be understood that any known therapy may be combined with embodiments of the present invention.

One aspect of embodiments of the present invention can be the administration of therapies that involve inducing at least one grief cycle (15). This grief cycle can relate to one or more specific events. Further embodiments can also involve inducing a secondary grief cycle (16) such as might be induced or elicited due to different types of stress. As mentioned above, this grief cycle may involve a grief cycle associated with accepting one's own death. It can also subsequently involve the grief cycle associated with other events perhaps such as accepting the fact that one did not die. Regardless of the particular therapy selected or utilized, the therapy can facilitate transformation of the intercranial communication pathway (1) or conceptual functionality. This can lead to the step of transforming an intercranial communication pathway (1), conceptually or actually, such as to provide enhanced communication bandwidth or the like so that functionality associated with a broader temporal perspective can be developed or used. The overall process can also involve merely accelerating the transformation such as in the step of accelerated transforming of the intercranial communication pathway (1).

After accomplishing the step(s) of administering a stress-related disorder therapy at an effective level, or even repetitive steps of administrating the particular therapy, a reassessment can occur. This can be such as the process of reassessing a particular parameter (19) such as one indicative of at least one intercranial communication level in order to track performance or treatment efficacy. Naturally, the step of reassessing a particular parameter (19) can involve the parameters utilized earlier. This may be particularly useful as an assessment of a concentrated emotional presentness level (26) or an assessment of an emotional intensity level (9) can be useful to show progress.

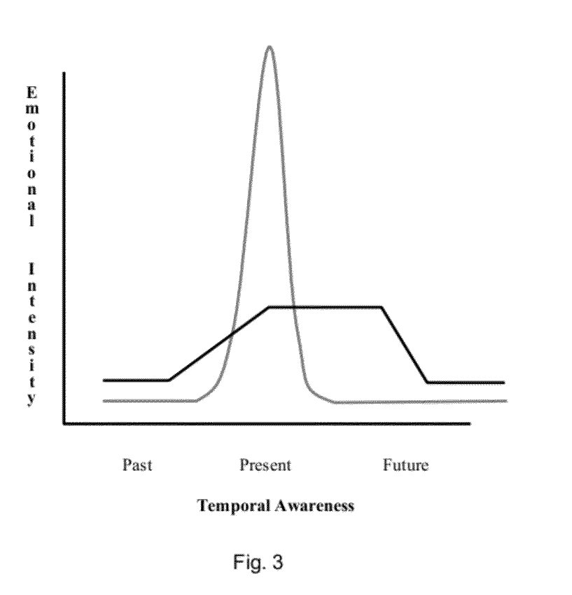

The aspect of showing progress can be accomplished through a display (73). The display (73) can also graphically display conceptual foundations that can be quite beneficial for the subject in the overall therapy, much like the mere temporal awareness generator as discussed above. For instance, the step of displaying cognitive information to aid in therapy (25) can be included. This display could be a quantitative or graphical measurement or estimate display or a more abstract display. A quantitative or graphical measurement or estimate display could include items such as a level of a concentrated temporal awareness, a level of emotional intensity, a change in such values or the like. An example of one type of a graphical display such as that shown in FIG. 3, which conceptually indicates a combination of temporal awareness along the horizontal axis and an emotional intensity along the vertical axis. As discussed above, it can be easily appreciated how with a more narrow present temporal awareness, emotional intensity can be dramatically increased if the so called volume of emotion is understood as a constant. It is perhaps the development of a more broad-based temporal awareness, such as can be considered from the perspective of transforming the intercranial communication pathway (1) that can indeed aid in reducing the emotional intensity at any given point in time. Of course, the display (73) can be configured to display a level or graphic or both indicative of an assessment of concentrated emotional presentness, and can serve as a computer generated concentrated emotional presentness graphical display. It may also be configured to display a level or graphic or both indicative of an assessment of emotional intensity, and as such can serve as a computer generated emotional intensity graphical display. Naturally, both a computer generated concentrated emotional presentness graphical display and a computer generated emotional intensity graphical display can be displayed to aid the subject. Comparison and progress displays can be used beneficially such as by including a computer generated precedent-subsequent concentrated emotional presentness graphical display and/or a computer generated precedent-subsequent emotional intensity graphical display.

An aspect that can be automated in embodiments of the invention can include the automated capture of information with an associated comparison, whether or not used to adjust the therapy or not. As shown in FIG. 1, the automated recording of subject treatment-precedent information (11) can be included as well as an automated recording of subject treatment-subsequent information (18). Furthermore, the step of comparing subject treatment-precedent information with subject treatment-subsequent information (20) can be used through an emotional presentness precedent-subsequent information comparator, a concentrated emotional presentness precedent-subsequent information comparator, or an emotional intensity precedent-subsequent information comparator. Through these, activity can be automated as an input to a potential step of adjusting a therapy administration (21) or just a step of indicating whether or not to adjust a therapy administration (21) from a particular perspective such as that of measuring concentrated temporal awareness, emotional intensity, combinations thereof, or the like. This automated process can provide indications from which a counselor (33) or even a subject (34) can understand and further evaluate the need to perhaps further revisit the events or even further experience grief cycles.

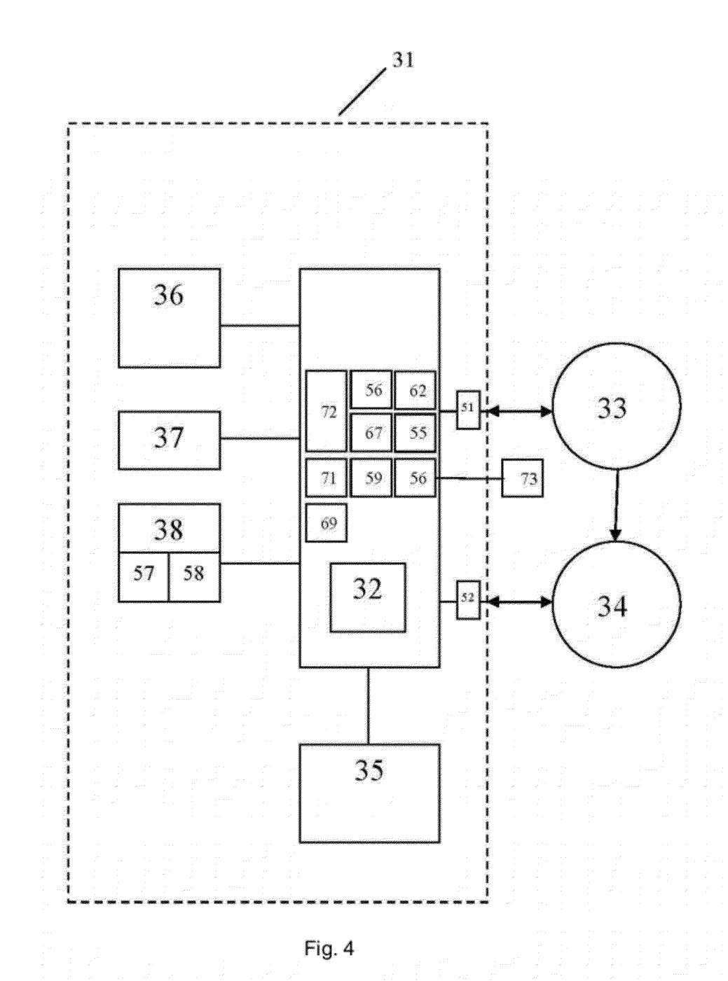

As shown in FIG. 4, devices can be used to implement various embodiments of the invention and the various processes available. As shown, an overall therapy aid (31), perhaps such as a pre-programmed computer or computer component, ASIC, or the like, can include a subject information input (51) to accept input from or relating to the subject (34) and then can process that input or at least aid in the overall therapy process. The aid can be software or programming in a computer or other programmed processor. Regardless as to how configured, this can involve providing data to or from a counselor (33) or other therapy provider such as through a therapist input (52). A counselor (33) can provide input or therapy to the subject (34). Within the therapy aid (31) or associated with it may be a recordation element (32), which can accept subject information such as subject treatment-precedent information (11), and thus serve as a subject treatment-precedent information recordation element, or may accept subject treatment-subsequent information (18) thus serve as a subject treatment-subsequent information recordation element. This information can be stored within a memory (36), a subject data information memory, or other similar functionality. Integral to the therapy aid (31) can be some type of comparison processor (38), which can be used to compare pre- and post information and can provide further indications such as to counselor (33) from which therapy can be adjusted. As such a system can include a concentrated emotional presentness precedent information memory, a concentrated emotional presentness subsequent information memory, an emotional intensity precedent information memory, and an emotional intensity subsequent information memory.

Adjustments can be programmed and automatically calculated to guide counselor (33) such as indicating a need to revisit a prior grief experience or the like. An important aspect to the therapy can also be utilization of a temporal awareness generator (37), which can provide to the subject (34) a graphical or perhaps value understanding such as that of a concentrated emotional presentness (26) or an emotional intensity (9). Importantly to some embodiments is the possibility that this temporal awareness generator (37) can provide a display (35) that can be used by the subject (34) or the counselor (33) to aid in understanding. For instance it has been seen that in some examples this simple visualization and understanding of the concept of concentrated emotional presentness (26) can greatly aid in the overall therapy, in participation in therapy, or even in return to therapy.

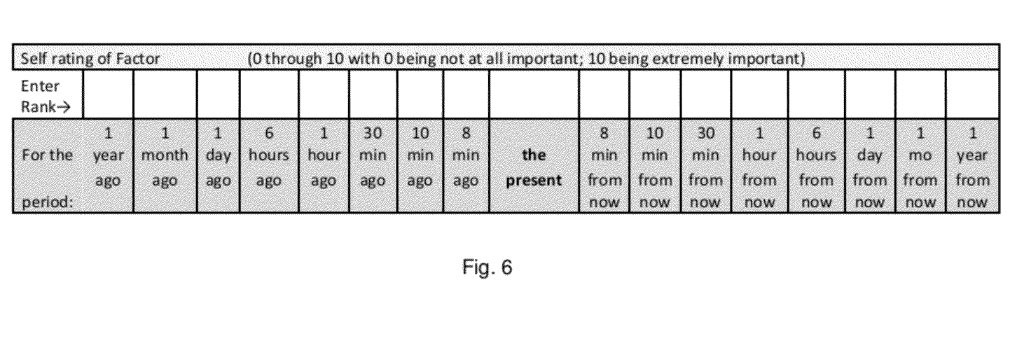

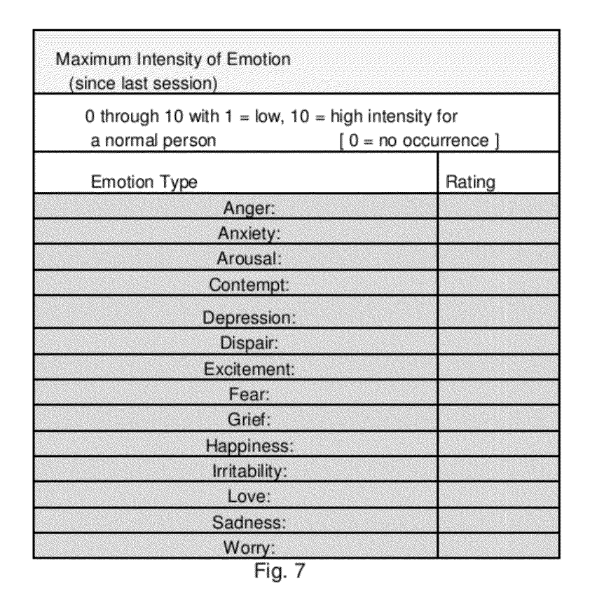

System elements that may be incorporated to act as elements to obtain subject treatment-precedent information (11) or subject treatment-subsequent information (18) can include: fill in forms (electronic and automatic, perhaps with data capture), speech analysis components (such as to access word occurrences or the like), self analysis elements, touch screens (such as to graphically rate color intensity aspects or the like), pressure sensors, brain scans, and other aspects. One example that can be easily understood may be that of a self evaluation form perhaps such as those shown in FIGS. 6 and 7. These forms can be used to glean information on the subject's self-perception of both Concentrated Emotional Presentness (CEP) and Emotional Intensity (EI). Of course all values, all windows, and all aspects can be varied as appropriate to any model. As shown, the subject can fill out these forms before and periodically during and after treatment or counseling. Analysis of the data can then be conducted.

Factors can be used to set relative perceived values such as in filling in all blanks, or might be used to simply set a particular level to a particular inquiry or factor. In setting a factor for estimation of CEP, some aspects that can be use can be abstract constructs, perhaps such as “Importance” or “Relevance” to the subject. Conversely, specific questions can be used to leave less to a subject person's interpretation. For example, two examples of the various types of questions that could be used for self-rating (separately or as a composite for the subject to answer, or as part of a single value or full ranking of all options) might include:
a) How far out/back can you think of something that would/did make you happy? and/or
b) How far out/back do you worry about something?

Interestingly, it may be noted that the CEP scale includes an interesting timeframe that might at first glance appear to present somewhat of an anomaly. Notice that the time frames of 10 minutes and 8 minutes are both used. These differ by only two minutes, of course. The present inventor's own personal experience with the issue is that the differences in time shown approximate a logarithmic scale, such as an earthquake scale where one level difference represents a ten-fold or such change in impact or risk. For example, the difference between a subject experiencing an 8 minute frame of reference versus a 10 minute frame of reference may be extremely significant. Indeed, the present inventor initially experienced the 8 minute level of CEP and eventually was able to get that “all the way” to 10 minutes as something viewed as great progress. There is little doubt that the normal population would not be likely respond to such questions with such a level of concentrated emotional presentness.

As mentioned above, the data gleaned may be analyzed such as by a temporal awareness generator (37). Mere awareness, perhaps especially graphically, can be significant. Such output and the software, subroutines, hardware, or processes of assessing subject information for maximum benefit can serve as an automated intercranial functionality transformation element (57) that causes, effects, suggests, or indicates actions to take to aid the subject. The temporal awareness generator (37) or an emotional intensity awareness generator may qualitative or quantitative. Embodiments may thus include a quantitative emotional presentness generator, a qualitative emotional presentness generator, a quantitative emotional intensity generator, a qualitative emotional intensity generator, an emotional presentness value generator, and/or an emotional intensity value generator. Quantitatively, embodiments may include statistical analysis elements, a subject input statistical analysis element, mathematical transformation elements, or other elements that assess the information available such as that provided by the forms of other subject treatment-precedent information gathering element or subject treatment-subsequent information gathering element. Non-limiting examples may include using statistical or other analysis functions such as skewness, such as through a data skew analysis element, or distribution functions, such as through a data distribution analysis element, perhaps all such as readily available in existing statistical analysis programs, Microsoft Excel, or other data software or database software. These functions can return in indication or even a quantitative value that is indicative of how present, how future leaning, or how past focused an individual appears to be, especially for multiple level ranking types of inquiries (i.e., fill in and rank all blanks versus just set the level for the subject a present). Such functions can also be applied with comparison to normal individuals so a comparison with a typical person (who may have a tendency to be more future oriented for example) to determine how affected the subject is. Thus for example if a typical individual has a skewness to the future with a value of, say +2.9*, whereas a subject has a skewness of −0.14* (indicating a high presentness orientation), a determination of propensity to, or current existence of PTSD impairment might be readily determined even prior to manifestation of problematic symptoms. Similarly, if a typical individual has a distribution (perhaps such as standard deviation or poisson distribution as non-limiting examples) about a relative maximum returning a value of, say +31*, whereas a subject has a distribution of +4* (also indicating a high presentness orientation), a determination of propensity to, or current existence of PTSD impairment might be readily determined in another, perhaps confirmatory, manner again even prior to manifestation of problematic symptoms. (* Numbers used only for example and not as actual data values since models applied can and will vary.)

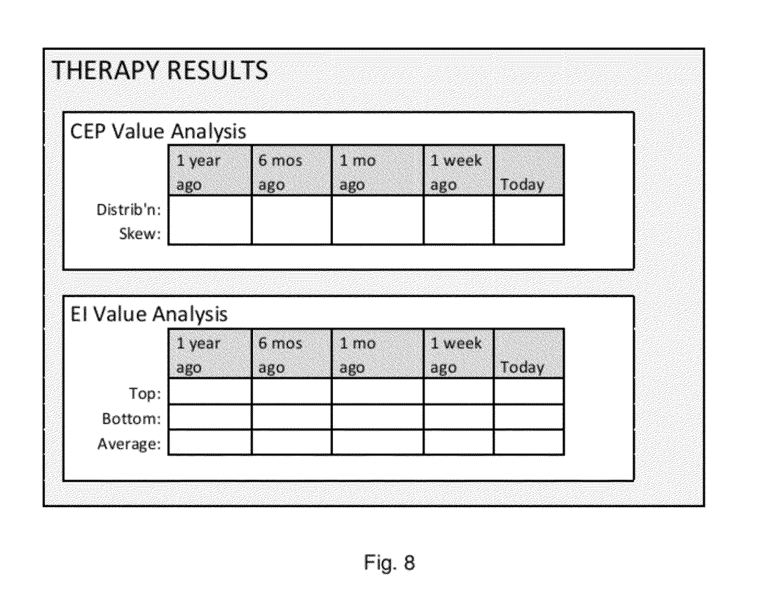

Of course, values can be used to rate an individual, as an evaluative tool, as a treatment threshold trigger, as well as to determine efficacy of an existing treatment, to motivate existing treatment progress, to encourage continued participation in an existing treatment, and for other reasons. An example of one type of display (35) is that of a composite quantitative value display as shown in FIG. 8. In these regards, embodiments may include a comparison processor (38) and a display (35). The comparison processor (38) could generate values. The display (35) could provide a subject or counselor with an indication from which to see progress, alter treatment processes, re-experience grief, or the like. Similarly, the emotional intensity values can identify areas for focus and treatment progress as well. Further both the CEP and EI aspect can be interrelated such as by weighting of values or other processes so that confirmation or other value analysis can occur.

In analyzing data a variety of processing elements can be used and so the system can include a computer generated assessment element (67). Values gleaned from the data may be inputted to a computer generated assessment element (67) perhaps configured as an emotional presentness generator (56) which can be an ASIC, hardware, software, or subroutines that process the data to generate a value indicative of a level of emotional presentness for the subject. The emotional presentness generator (56) can use any of the above mentioned analysis processes or other types of processes whether complex or simple (such as merely using an indicated value for a single answer question) and may yield some indication of the subject's CEP level and thus serve as a concentrated emotional presentness information generation element. Similarly, embodiments may include an emotional intensity generator (59) which can be an ASIC, hardware, software, or subroutines that process the data to generate a value indicative of a level of emotional intensity for the subject. The emotional intensity generator (59) can use any of the above mentioned analysis processes or other types of processes whether complex or simple (such as merely using an indicated highest value for a single emotion or a constant emotion value) to yield some indication of the subject's EI level. By including initial parameter storage (57) and subsequent parameter storage (58) values can be compared automatically perhaps by proper configuring of the computer generated assessment element (67).

The computer generated assessment element (67) can be applied multiple times in the course of various treatments and can thus serve as a parameter reassessment element (69) to evaluate the efficacy of treatment, show concrete results, or the like. In evaluating treatment results, the system may include a therapy transformation processor (72) to alter a therapy for appropriate effect. The therapy transformation processor (72) can provide input to the automated intercranial functionality transformation element (57) to provide optimal, better, or just changed input to alter the therapy to check for a positive or better intercranial functionality transformation. By having the system provide input to treatment provider or by providing the treatment itself, the system can include a stress-related disorder therapy indicator (62) which can even simply output a stress-related disorder therapy indication or guidance. This can cause or indicate changes in treatment or the like based on the results shown. Re-treatment or multiple treatments can be indicated and embodiments can include a multiple treatment intercranial functionality transformation element.

In changing treatment or causing others to change treatment, the system can include a therapy adjustment element (71). This can act by assessing quantitatively or qualitatively results indicated and acting or indicating accordingly. These adjustments could include determining that it may be appropriate to induce or re-induce a grief process and as such the system may include a grief process institution element (55) or even a secondary grief process institution element (56) as two examples of various treatment processes that may be indicated, induced, or re-induced. Other processes or elements can be included ranging from a death acceptance process, a death acceptance process institution element, and even a life acceptance process, or a life acceptance process institution element. Of importance is that the system is conceptually, metaphorically, or actually desired to achieve an intercranial transformation that is automated to some degree perhaps such as to diminish the CEP or EI levels of the subject. The combination of elements and individual programming, hardware, or subroutines can provide an automated intercranial functionality transformation element (57) that causes treatment or provides directly treatment(s) that help speed or cause the transformation to help the subject return to a more normal level of functioning.

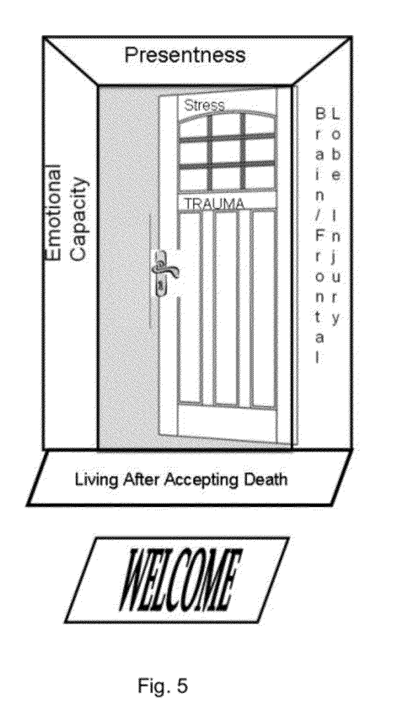

Referring now to FIG. 5, it can be conceptually understood how alternative embodiments can use differing displays (35) that are both quantitative as well as merely conceptual. FIG. 5 shows activities of the present process for visualization in differing conceptual frameworks. For example as show in FIG. 5, the subject (34) can be presented a perspective of passing through a doorway with involves all of the aspects of emotional intensity or capacity (9), the sense of presentness, the aspects of intercranial lobe injury, as well as the aspect of returning to normal function after having accepted the fact that one would die. Passing through the doorway can be to face the stress or trauma event in order to ameliorate its effects. As examples of actual applications of the present invention have and are anticipated to continue to show, such mental images can significantly enhance the therapy effectiveness and results.

As can be understood from the foregoing, the basic concepts of the present invention may be embodied in a variety of ways. It involves both therapy techniques as well as devices to accomplish the appropriate therapy. In this application, the therapy techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in device-oriented terminology, each element of the device implicitly performs a function. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing both the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “recordation element” or “recorder” should be understood to encompass disclosure of the act of “recording”—whether explicitly discussed or not—and, conversely, were there effectively disclosure of the act of “recording”, such a disclosure should be understood to encompass disclosure of a “recorder,” a “recordation element,” and even a “means for recording.” Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function.

Any patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed below are hereby appended and hereby incorporated by reference, however, as to each of the above, to the extent that such information or statements incorporated by reference might be considered inconsistent with the patenting of this/these invention(s) such statements are expressly not to be considered as made by the applicant(s).

REFERENCE TABLE
I. US PATENT AND PATENT PUBLICATION DOCUMENTS

| DOCUMENT NO. & | | |
|---|---|---|
| KIND CODE | PUBLICATION/ISSUE | PATENTEE OR |
| (if known) | DATE mm/dd/yyyy | APPLICANT NAME |
| 20100010371 A1 | 2010-01-14 | Zayfert et al. |
| 7,565,193 B2 | 2009-07-21 | Laken |
| 7,410,493 B1 | 2008-08-12 | Chen et al. |
| 7,381,747 B2 | 2008-06-03 | Dooley et al. |
| 7,353,065 B2 | 2008-04-01 | Morrell |
| 6,689,816 B2 | 2004-02-10 | Fogel |

II. NON-PATENT LITERATURE
Document

Adshead, G., Psychological therapies for post-traumatic stress disorder,
British Journal of Psychiatry 2000 177; pp. 144-148
Freeman, C., Psychological and drug therapies for post-traumatic
stress disorder, Psychiatry, Volume 5, Issue 7, (July 2006) pp. 231-237
Bryant, R. A. et al., Hypnotherapy and cognitive behaviour therapy of
acute stress disorder: a 3-year follow-up, Behaviour Research and
Therapy 44 (2006) pp. 1331-1335
Gillespie, K. et al., Community based cognitive therapy in the treatment
of post-traumatic stress disorder following the Omagh bomb, Behaviour
Research and Therapy 40 (2002) pp. 345-357
Ehlers, A. et al., Cognitive therapy for post-traumatic stress disorder:
development and evaluation, Behaviour Research and Therapy 43
(2005) pp. 413-431
Sarid, O. et al., Trauma and acute stress disorder: A comparison
between cognitive behavioral intervention and art therapy, The Arts
in Psychotherapy, Volume 37, Issue 1, February 2010, pp. 8-12
Hoge, C. M. et al., Combat Duty in Iraq and Afghanistan, Mental Health
Problems, and Barriers to Care, N Engl J Med 2004; 351: 13-22
Hoge, C. M., et al. Mental Health Problems, Use of Mental Health
Services, and Attrition From Military Service After Returning From
Deployment to Iraq or Afghanistan, JAMA. 2006; 295(9): 1023-1032.
doi: 10.1001/jama.295.9.1023
Dohrenwend, B. P., et al. The Psychological Risks of Vietnam for
U.S. Veterans: A Revisit with New Data and Methods, Science 18
Aug. 2006: Vol. 313 no. 5789 pp. 979-982 DOI: 10.1126/
science.1128944
Prigerson H. G. et al. Traumatic grief as a distinct disorder. In: Stroebe
MS, Hansson RO, Stroebe W, Schut H, editors. Handbook of
bereavement research: consequences, coping, and care. New York:
APA Press; 2001. pp. 613-645
Litz, B. et al. Early cognitive-behavioral interventions for adults. In E.
Foa, T. Keane, M. Friedman, J. Cohen, & (Eds.), Effective treatments for
PTSD: Practice guidelines from the International Society for Traumatic
Stress (2009). (pp. 117-135). New York: Guilford Press.
Straits-Troster K, et al., An Adaptation of Multi-Family Group Treatment
to Support U.S. Veteran TBI Survivors and their Families. Brain
Impairment, 2011; 12: S15-16
Provisional Patent Application Number 61/432,073, filed Jan. 12, 2011

Thus, the applicant(s) should be understood to have support to claim and make a statement of invention to at least: i) each of the methods or processes as herein disclosed and described, ii) the related devices disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

In addition and as to computer aspects and each aspect amenable to programming or other electronic automation, the applicant(s) should be understood to have support to claim and make a statement of invention to at least: xv) processes performed with the aid of or on a computer as described throughout the above discussion, xvi) a programmable apparatus as described throughout the above discussion, xvii) a computer readable memory encoded with data to direct a computer comprising means or elements which function as described throughout the above discussion, xviii) a computer configured as herein disclosed and described, xix) individual or combined subroutines and programs as herein disclosed and described, xx) a carrier medium carrying computer readable code for control of a computer to carry out separately each and every individual and combined method described herein or in any claim, xxi) a computer program to perform separately each and every individual and combined method disclosed, xxii) a computer program containing all and each combination of means for performing each and every individual and combined step disclosed, xxiii) a storage medium storing each computer program disclosed, xxiv) a signal carrying a computer program disclosed, xxv) the related methods disclosed and described, xxvi) similar, equivalent, and even implicit variations of each of these systems and methods, xxvii) those alternative designs which accomplish each of the functions shown as are disclosed and described, xxviii) those alternative designs and methods which accomplish each of the functions shown as are implicit to accomplish that which is disclosed and described, xxix) each feature, component, and step shown as separate and independent inventions, and xxx) the various combinations and permutations of each of the above.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 20 or any other claim” or the like, it could be re-drafted as dependent on claim 1, claim 15, or even claim 25 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

1. An automated, computer process implemented system to aid in therapeutically treating incidents of stress-related disorders comprising:
a subject information input;
a computer generated assessment element responsive to said subject information input;
an automated intercranial functionality transformation element responsive to said computer generated assessment element; and
a stress-related disorder therapy indicator responsive to said automated intercranial functionality transformation element to institute a desired effect on a subject.

2. An automated, computer process implemented system to aid in therapeutically treating incidents of stress-related disorders as described in claim 1 wherein said computer generated assessment element is configured to assess an initial parameter indicative of at least one intercranial functionality level.

3. An automated, computer process implemented system to aid in therapeutically treating incidents of stress-related disorders as described in claim 1 and further comprising:
a subject treatment-precedent information recordation element;
a subject treatment-subsequent information recordation element;
a therapy transformation processor responsive to said subject treatment-precedent information recordation element and said subject treatment-subsequent information recordation element.

4. An automated, computer process implemented system to aid in therapeutically treating incidents of stress-related disorders as described in claim 3 wherein said computer generated assessment element comprises an concentrated emotional presentness information generation element.

5. An automated, computer process implemented system to aid in therapeutically treating incidents of stress-related disorders as described in claim 4,
wherein said subject treatment-precedent information recordation element comprises a concentrated emotional presentness precedent information memory,
wherein said subject treatment-subsequent information recordation element comprises a concentrated emotional presentness subsequent information memory, and
wherein said therapy transformation processor comprises a concentrated emotional presentness precedent-subsequent information comparator.

6. An automated, computer process implemented system to aid in therapeutically treating incidents of stress-related disorders as described in claim 3 wherein said computer generated assessment element comprises an emotional intensity information generation element.

7. An automated, computer process implemented system to aid in therapeutically treating incidents of stress-related disorders as described in claim 6,
wherein said subject treatment-precedent information recordation element comprises an emotional intensity precedent information memory,
wherein said subject treatment-subsequent information recordation element comprises an emotional intensity subsequent information memory, and
wherein said therapy transformation processor comprises an emotional intensity precedent-subsequent information comparator.

8. An automated, computer process implemented system to aid in therapeutically treating incidents of stress-related disorders as described in claim 5 wherein said computer generated assessment element further comprises an emotional intensity information generation element.

9. An automated, computer process implemented system to aid in therapeutically treating incidents of stress-related disorders as described in claim 1 wherein said automated intercranial functionality transformation element comprises a temporal awareness generator.

10. An automated, computer process implemented system to aid in therapeutically treating incidents of stress-related disorders as described in claim 9 wherein said temporal awareness generator comprises a computer generated display.

11. An automated, computer process implemented system to aid in therapeutically treating incidents of stress-related disorders as described in claim 10 wherein said computer generated display comprises a computer generated concentrated emotional presentness graphical display.

12. An automated, computer process implemented system to aid in therapeutically treating incidents of stress-related disorders as described in claim 5 and further comprising a computer generated precedent-subsequent concentrated emotional presentness graphical display.

13-17. (canceled)

18. An automated, computer process implemented system to aid in therapeutically treating incidents of stress-related disorders as described in claim 1 wherein said computer generated assessment element comprises an emotional presentness information generator.

19. An automated, computer process implemented system to aid in therapeutically treating incidents of stress-related disorders as described in claim 18 wherein said emotional presentness information generator comprises a quantitative emotional presentness generator.

20. An automated, computer process implemented system to aid in therapeutically treating incidents of stress-related disorders as described in claim 19 wherein said quantitative emotional presentness generator comprises a concentrated emotional presentness value generator.

21. An automated, computer process implemented system to aid in therapeutically treating incidents of stress-related disorders as described in claim 20 wherein said concentrated emotional presentness value generator comprises a subject input statistical analysis element.

22. An automated, computer process implemented system to aid in therapeutically treating incidents of stress-related disorders as described in claim 21 wherein said subject input statistical analysis element comprises a data distribution analysis element.

23. An automated, computer process implemented system to aid in therapeutically treating incidents of stress-related disorders as described in claim 21 wherein said subject input statistical analysis element comprises a data skew analysis element.

24. (canceled)

25. An automated, computer process implemented system to aid in therapeutically treating incidents of stress-related disorders as described in claim 1 wherein said computer generated assessment element comprises an emotional intensity information generator.

26. An automated, computer process implemented system to aid in therapeutically treating incidents of stress-related disorders as described in claim 25 wherein said emotional intensity information generator comprises a quantitative emotional intensity generator.

27-34. (canceled)

35. An automated, computer process implemented system to aid in therapeutically treating incidents of stress-related disorders as described in claim 34 and further comprising a precedent-subsequent information comparator.

36. An automated, computer process implemented system to aid in therapeutically treating incidents of stress-related disorders as described in claim 35 and further comprising a therapy transformation processor responsive to said precedent-subsequent information comparator.

37. An automated, computer process implemented system to aid in therapeutically treating incidents of stress-related disorders as described in claim 1 wherein said stress-related disorder therapy indicator comprises a grief process institution element.

38. An automated, computer process implemented system to aid in therapeutically treating incidents of stress-related disorders as described in claim 37 wherein said stress-related disorder therapy indicator comprises a secondary grief process institution element.

39. An automated, computer process implemented system to aid in therapeutically treating incidents of stress-related disorders as described in claim 37 wherein said stress-related disorder therapy indicator comprises a death acceptance process institution element.

40. An automated, computer process implemented system to aid in therapeutically treating incidents of stress-related disorders as described in claim 39 wherein said stress-related disorder therapy indicator comprises a life acceptance process institution element.

41. A computer implemented process to aid in therapeutically treating incidents of stress-related disorders comprising the steps of:
inputting subject information to an automated system;
computer generating an assessment of said subject information;
automatically determining a desirable process to attempt to transform an intercranial functionality; and
outputting a stress-related disorder therapy indication to institute a desired effect on a subject.

42. A computer implemented process to aid in therapeutically treating incidents of stress-related disorders as described in claim 41 wherein said step of computer generating an assessment of said subject information comprises the step of computer generating a temporal assessment of said subject information.

43. A computer implemented process to aid in therapeutically treating incidents of stress-related disorders as described in claim 42 wherein said step of computer generating a temporal assessment of said subject information comprises the step of computer generating an emotional presentness assessment of said subject information.

44. A computer implemented process to aid in therapeutically treating incidents of stress-related disorders as described in claim 43 wherein said step of computer generating an emotional presentness assessment of said subject information comprises the step of computer generating a concentrated emotional presentness assessment of said subject information.

45. A computer implemented process to aid in therapeutically treating incidents of stress-related disorders as described in claim 41 wherein said step of computer generating an assessment of said subject information comprises the step of computer generating an emotional intensity assessment of said subject information.

46. A computer implemented process to aid in therapeutically treating incidents of stress-related disorders as described in claim 44 wherein said step of computer generating an assessment of said subject information further comprises the step of computer generating an emotional intensity assessment of said subject information.

47. A computer implemented process to aid in therapeutically treating incidents of stress-related disorders as described in claim 43 wherein said step of computer generating an emotional presentness assessment of said subject information comprises the step of computer generating a quantitative concentrated emotional presentness assessment of said subject information.

48. A computer implemented process to aid in therapeutically treating incidents of stress-related disorders as described in claim 47 wherein said step of computer generating an assessment of said subject information further comprises the step of computer generating a quantitative emotional intensity assessment of said subject information.

49. A process for therapeutically treating manifestations of stress-related disorders comprising the steps of:
a. establishing an external condition conducive to an enhanced rate of intercranial functionality modeled as an intercranial communication pathway;
b. assessing an initial parameter indicative of at least one intercranial communication level;
c. administering a stress-related disorder therapy at effective levels; and
d. acceleratedly transforming said intercranial communication pathway from enhanced communication bandwidth.

50-86. (canceled)
